(19) United States
(12) Patent Application Publication
(10) Pub. No.: US 2023/0088251 A1
(43) Pub. Date: Mar. 23, 2023

(54) LIGHT-ACTIVATED COATING AND MATERIALS

(71) Applicant: ChromaSol International LLC, Roswell, GA (US)

(72) Inventors: William Clary Floyd, Chester, SC (US); Alan P. Kozikowski, Wallace, WI (US); Peter Stubbs, York (GB)

(21) Appl. No.: 17/980,802

(22) Filed: Nov. 4, 2022

Related U.S. Application Data
(63) Continuation-in-part of application No. 16/088,059, filed on Sep. 24, 2018, now Pat. No. 11,498,983.

(51) Int. Cl. A61L 31/16

(52) U.S. Cl. CPC A61L 31/16; A61L 2300/442; A61L 2300/30; A61L 2300/216

(57) ABSTRACT

A surgical type face mask having a substrate forming an outer layer. The outer layer being treated with a water-based polymer containing at least one photosensitizer in sufficient quantity to impart a characteristic color. In the presence of visible light, the water based polymer generates therapeutically useful quantities of singlet oxygen for treatment, by inhalation thereof, of respiratory infections and cancers.

### CROSS-REFERENCE TO RELATED APPLICATION

This application is a Continuation In Part of, and claims priority to and the benefit of, U.S. patent application Ser. No. 16/088,059 filed on Sep. 24, 2018, which claims priority to and the benefit of PCT/US2017/024162 filed Mar. 24, 2017, which claims priority to and the benefit of U.S. patent application Ser. No. 62/313,231 filed on Mar. 25, 2016, the entirety of which is incorporated herein by reference.

### TECHNICAL FIELD

This disclosure relates to temporary coatings having light-activated properties, and methods for applying and removing temporary coatings. This application relates to coatings for surfaces and more particularly to anti-microbial coatings. This application also provides compositions and methods for preventing and treating infections and has applications in the areas of medicine, pharmacology, virology, and medicinal chemistry.

### BACKGROUND

There are very few good options for preventing or treating infections, including “common colds.” In the prior art no fast working and efficient composition has been provided for preventing and/or treating common colds initiated by viral infections caused by the cold viruses, such as rhino virus, corona virus, adenovirus, coxsackie virus, RS-virus, echovirus or other cold viruses yielding the usual well known cold syndromes in patients. Practically all humans suffer 2 to 3 times a year from infections in the upper respiratory passages, such as cold and flu. In general, the majority of common colds occurring in September, October and November are caused by rhinovirus infection, whereas the majority of common cold occurring in January, February and March are caused by Coronavirus infections. Furthermore, there is a great need for effective remedies in the increasing number of patients suffering from allergic syndromes, for example asthma, which may be initiated by common cold viruses, especially the rhinovirus.

Rose Bengal has long been known in the biological community as a staining agent for tissue. It has been used in eye drops to stain damaged corneal tissue. In synthetic organic chemistry it has been used as a photocatalyst to generate singlet oxygen, which is the electronically excited state of ordinary molecular oxygen. Singlet oxygen has a short half life under various conditions ranging from microseconds to several minutes. Ifs half-life in various solvents ranges from a few microseconds to a few milliseconds. In ambient air, it has been reported to be a few seconds. In the upper atmosphere, it has been reported as minutes in air. Its half-life in solvents is milliseconds/nanoseconds, but has been reported in minutes in air. Singlet oxygen rapidly decays back to ordinary ground-state triplet oxygen by reacting with other molecular species and transferring its excitation energy or undergoing a chemical reaction. This singlet oxygen has also shown a sanitizing effect in eliminating harmful bacteria and viruses. Singlet oxygen generated from numerous photocatalytic sources has been shown to be a safe and effective antimicrobial technology. Delivery and associated hazardous effects have created issues with the use and delivery of singlet oxygen.

Accordingly, there is always a need for improved methods and systems for preventing the spread and treating infections. It is to those needs, among others, that this application is directed.

### SUMMARY

One aspect of this application photosensitizers have been applied to a fabric substrate, such as a mask for covering the mouth and nose of a user, by means of a water-based polymer. The photosensitizers are present in an amount sufficient to impart a characteristic color to the substrate, and in the presence of visible light, catalyze formation of singlet oxygen in therapeutically useful quantities for treating respiratory infections and cancers by inhalation of the singlet oxygen. It is believed that oxidative interaction of singlet oxygen with external functionality of pathogens may make the pathogen's antigens more accessible to the body's immune system, eliciting an immune response against it.

In a preferred embodiment the photosensitizer is a polymer having a first monomer operatively connected to dye (e.g., Rose Bengal), a second monomer, and a surfactant. The surfactant is selected from the group of ionic surfactants, anionic surfactants, cationic surfactants, nonionic surfactants, zwitterionic surfactants, and mixtures thereof. The Rose Bengal or the dye in polymer in an amount effective for rendering the polymer antimicrobial or antiviral upon exposure of the polymer to light; and the polymer produces singlet oxygen from air in the presence of light.

Another aspect of this application is a method of treating an immune reaction in a mammalian or avian species. The method includes administering locally (one or more times) to nasal passages of the mammal or fowl singlet oxygen from a coating. The coating has a polymer of a first monomer operatively connected to a dye (e.g., Rose Bengal), a second monomer, and a surfactant, wherein the surfactant is selected from the group consisting of ionic surfactants, anionic surfactants, cationic surfactants, nonionic surfactants, zwitterionic surfactants, and mixtures thereof. The Rose Bengal in the polymer is in an amount effective for rendering the polymer antimicrobial or antiviral upon exposure of said substrate to light; and the polymer produces singlet oxygen from air in the presence of light.

### FIGURES

FIG. 1 represents illustrative examples of a first monomer connected to a dye (e.g., Rose Bengal).

### DETAILED DESCRIPTION

The mask constructed in accordance with the invention provides a mechanism for Photodynamic Singlet Oxygen Inhalation Immunotherapy. Specific embodiments of this disclosure include a polymer-bound version of Rose Bengal and other dyes. The Rose Bengal molecule, e.g., may be modified to incorporate functionality, of the Rose Bengal, into a molecule or monomer that may be polymerized or co-polymerized with cationic, nonionic or anionic monomers (e.g., vinyl and acrylic monomers). In specific embodiments, the monomers may be polymerized by using free-radical polymerization. Both an aqueous solution polymer and an aqueous emulsion polymer incorporating the monomers have been developed.

Accordingly, in one aspect, the present teaching provides a process for the preparation of an antimicrobial coating solution, the process of polymerizing certain monomers and dyes to form a coating suitable for application to a surface. The coating can be placed on a substrate/surface as a thin homogeneous coating to be applied to a substrate (in this context, the term “thin” can mean approximately 10 nm to 400 nm thickness for a single layer) and still provide for effective antimicrobial action.

One or more of the foregoing dyes is effective against one or more of the following bacteria: Escherichia coli, Pseudomonas aeroginosa, Enterobacter cloacae, Staphylococcus aureus, Enterococcus faecalis, Klebsiella pneumoniae, Salmonella typhimurium, Staphylococcus epidermidis, Serratia marcescens, Mycobacterium bovis (TB), methicillin resistant Staphylococcus aureus and Proteus vulgaris. In addition, the foregoing dyes are effective against viruses, particularly enveloped viruses, such as Herpes, HIV, H5N1 (“bird flu”), and viruses associated with the “common cold.”

The first monomer for use with specific embodiments is operatively linked to a dye that absorbs light, becomes excited and transfers that excitation energy to molecular oxygen (O2), exciting it from the electronic ground state triplet to the excited singlet state. The dye acts as a light-activated catalyst for generating singlet oxygen. The singletoxygen is highly reactive and disables virus and bacteria on contact. One example is the alkali metal salts of Rose Bengal, 4,5,6,7-tetrachloro-2′,5′,7′-tetraiodo fluorescein sodium or potassium.

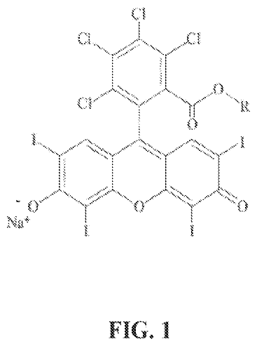

Several dyes have been examined and modified to allow their incorporation into polymers via free radical polymerization. FIG. 1 shows a representative example of a dye connected to a first monomer, wherein the R represents a stryenic group, an acrylic group, or another covalently bound group capable of further polymerization or copolymerization. In one example, R can be ortho, meta or para vinyl benzyl, ethyl acrylate, ethyl methacrylate, ethyl crotonate, ethyl acrylamido, ethyl methacrylamido. That is, e.g.,

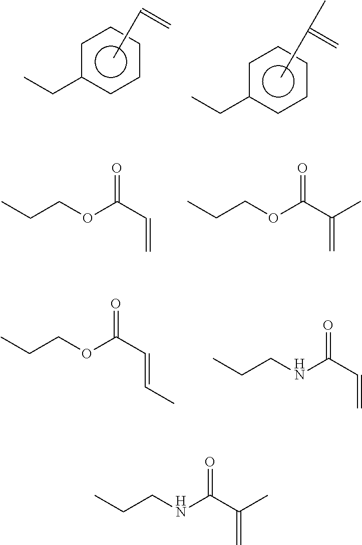

Rose Bengal has been the dye of choice for various reasons, and has been modified to contain either a styrenic or acrylic handle. In certain examples, a styrene group or an acrylic group was added to the carboxylic acid group of Rose Bengal. For example:

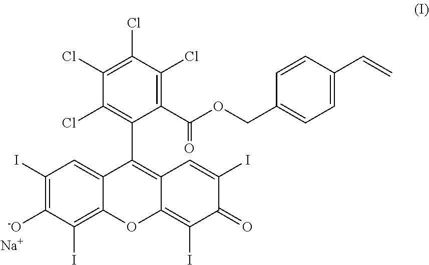

For another example:

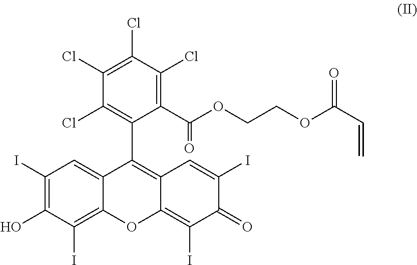 [SMILES: C=CC(=O)OCCOC(=O)c1c(Cl)c(Cl)c(Cl)c(Cl)c1-c1c2cc(I)c(=O)c(I)c-2oc2c(I)c(O)c(I)cc12]

For another example.

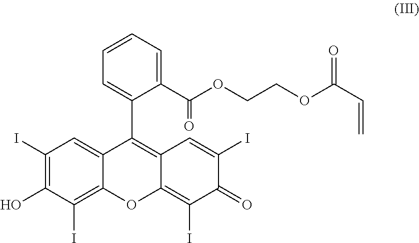 [SMILES: C=CC(=O)OCCOC(=O)c1ccccc1-c1c2cc(I)c(=O)c(I)c-2oc2c(I)c(O)c(I)cc12]

For another example,

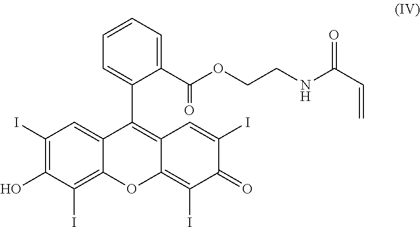 [SMILES: C=CC(=O)NCCOC(=O)c1ccccc1-c1c2cc(I)c(=O)c(I)c-2oc2c(I)c(O)c(I)cc12]

For another example,

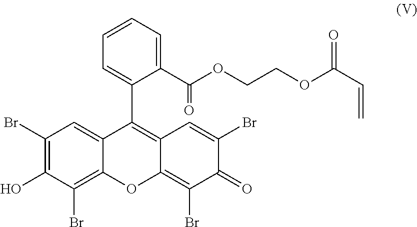 [SMILES: C=CC(=O)OCCOC(=O)c1ccccc1-c1c2cc(Br)c(=O)c(Br)c-2oc2c(Br)c(O)c(Br)cc12]

For another example,

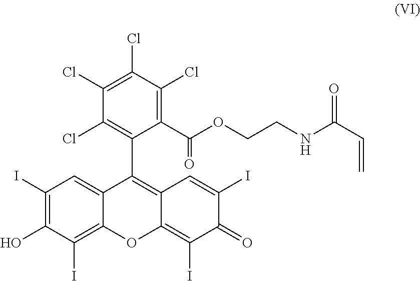 [SMILES: C=CC(=O)NCCOC(=O)c1c(Cl)c(Cl)c(Cl)c(Cl)c1-c1c2cc(I)c(=O)c(I)c-2oc2c(I)c(O)c(I)cc12]

In certain embodiments, the polymer may contain styrenic and acrylic Rose Bengal derivatives, which have been copolymerized with various vinyl and acrylic monomers. Rose Bengal, is a synthetic dye, 2,4,5,7-tetraiodo-3′, 4′, 5′, 6′-tetrachlorofluorescein. Rose Bengal is a typical photosensitizer to produce singlet oxygen, which can be used to kill microbes. Rose Bengal has many derivatives. The most common one is the sodium salt form, which is highly water soluble.

A solution polymer and an emulsion polymer have been developed for various applications. The solution polymer comprised various water-soluble monomers such as acrylic acid, itaconic acid and hydroxyethyl acrylate. It also can incorporate co-polymerizable surfactants such as allyl alcohol/1O EO or lauryl alcohol polyoxyethylene ammonium sulfate with a pendant allyl group. These surfactants were used to lower surface tension to enable the polymer to flow and wet the fiber, providing more surface area for exposure to air. The hydroxyl groups and acid groups could also cross-link upon curing to provide durability to the substrate. That is, in some examples, the polymer may dry as a hard coating.

A visible light activated antimicrobial coating composition is obtained by the monomers described herein. The coating described herein can be used under indoor or outdoor lighting conditions. The antimicrobial coating composition exhibits antimicrobial activity under visible light and in reduced light.

An exemplary emulsion polymer was designed for durability and water resistance, but also removability. Removability was achieved by incorporation of a large number of carboxylic acid groups at low pH (3-5), then use of an alkaline remover solution. Efficiency was achieved by means of core/shell architecture, in which the Rose Bengal monomer was incorporated predominantly in the shell of the particle, keeping it on the surface and not embedded deep within the polymer film.

Generally, long chain polymer molecules are synthetized by two types of polymerization, step growth and chain growth. Step growth is based on chemical reactions such as esterification or amidation. Chain growth is an alternative way to get long chain polymers. Chain growth is based on the opening of unsaturated bonds on monomers by initiators. Compared with step growth, chain growth more easily generates high molecular weight polymers. Many functional polymers can be polymerized by chain copolymerization.

Polymerization of the monomers in the emulsion can be effected by a suitable initiation system, for example, UV initiator, redox, or thermal initiator.

Substrates treated with either type of polymer containing a dye, e.g., the Rose Bengal monomer or polymer containing the monomer become self-sanitizing upon exposure to light. This has been proven with fabric used for medical fabrics, kitchen wipes, filter media and sportswear. The emulsion polymer has been evaluated as a spray on floors, carpets, towels and garments as a means of reducing or eliminating odor from bacterial activity. It has also been evaluated in floor wax for providing self-sanitizing floor surfaces. It has also been proposed as a treatment for faucet handles and push plates in rest rooms and commonly touched surfaces such as stair railings and grocery cart handles.

Specific embodiments include a coating suitable to provide an antimicrobial surface that is one that presents an antimicrobial agent that inhibits or reduces the ability of microorganisms to grow. Antimicrobial agents are agents that kill microorganisms or inhibit their growth. Antimicrobial agents can be classified by the microorganisms that they act against. For example, antibacterials are used against bacteria, anti-fungals are used against fungi and anti-virals are used against viruses. Such surfaces are desirable to prevent the spread of infection and so are desirable in healthcare settings such as hospitals, hospices, retirement homes and clinics, for example. While a material may or may not be inherently antimicrobial, the present application is directed generally to surfaces which do not possess inherent or sufficient antimicrobial properties and require a surface treatment or coating to become antimicrobial.

Another embodiment includes a polymer obtained by selecting a first monomer; operatively linking the first monomer to a dye, e.g., Rose Bengal or operatively incorporating the Rose Bengal, selecting a second monomer, polymerizing the first monomer and the second monomer using free radical polymerization. More than 50 percent of the first monomer can be linked to the Rose Bengal. The polymer may have less than 2% by weight of the first Rose Bengal monomer. The polymer may have less than I % by weight of the Rose Bengal operatively connected to the first monomer.

Any conventional process for making emulsion polymers may be suitable for preparing specific embodiments. Generally, latex emulsion polymers can be prepared by mixing the acid monomers with the hydrophobic monomers and surfactant together to form a monomer mixture. For example, emulsification can occur readily with mixing hydrophobic monomers and surfactant in water. Typically, a monomer mixture can be prepared by charging water and dissolving surfactant in the water. Acid monomers and hydrophobic monomers can then be added. The homogenization can be optionally facilitated by the use of homogenizing equipment and/or non-copolymerizable surfactants (e.g. ethoxylates) compatible with the temporary composition. A surfactant or surfactants can then be added to the monomer mixture and stirred to form an emulsion. The monomers are mixed with water and the copolymerizable surfactants to form a pre-emulsion, and then the monomers can be “stirred” to mix.

Alternatively, monomers and copolymerizable surfactants can be mixed (e.g. without water to form a monomer mixture). Often anionic surfactants or aqueous solutions of surfactants will not dissolve in pure monomer. The monomer pre-emulsion can be prepared from water, surfactant, acid monomers and hydrophobic monomers by any conventional means that is suitable, depending on the requirements of the specific components.

The surfactant(s) may include a copolymerizable surfactant, a noncopolymerizable surfactant, or a combination of copolymerizable and noncopolymerizable surfactants. In one embodiment, noncopolymerizable surfactants can be used to form the latex particle. The many parameters of emulsion polymerization techniques can be adjusted by those skilled in the art to obtain particular results such as particle size or freeze-thaw resistance. The monomers can be added to the aqueous phase gradually or in one charge. Monomers can be added continuously or in staggered finite increments.

Other ingredients can be added, as long as they are not deleterious to the antimicrobial and/or antiviral activity of the dye. For example, the singlet oxygen should not be quenched.

One specific embodiment includes copolymerizable surfactants, due to performance benefits from them being incorporated into the polymer. For example, the inability to migrate to the surface can reduce re-wetting and water-sensitivity. In one specific embodiment, the temporary coating prepared using copolymerized surfactants produced significantly less foam during the removal process as compared to traditional coatings. The reduction in foam can minimize slip hazards and allow for improved flocculation in waste water disposal.

The temporary coating may include a balanced formulation of hard monomers, soft monomers and copolymerizable surfactants to achieve a desired glass transition temperature (Tg). In one example, the monomers were balanced to achieve a Tg of −10 degrees C. Further, in other examples, the overall Tg of the polymer ranged from about 10 to 20 degrees C.

The polymers containing this Rose Bengal dye monomer have been applied to fabric of various types. Nonwoven nylon substrates are preferred. The polymer is applied at dry add-on levels of around 0.5%, giving the dried, cured fabric a pink color. Aging studies under continuous light show effective performance for at least 3 months. At 6 months performance may start to drop off The dye photodegrades eventually and the pink color fades to a light beige. At this point there is less antimicrobial activity. There is also no anti-microbial activity on the pink fabric in the dark. Fabric permanently bonded with the solution polymer has been subjected to over 25 simulated home launderings with less than 10 ppm leaching of the dye-containing polymer.

Other optional ingredients include, water, an amount of a suitable noncopolymerizable surfactant, thickeners, hiding pigments, opacifiers, colorants, antioxidants, biocides or any other ingredients typically added to latex polymers. Optional ingredients include conventional dispersants. These additional ingredients are not critical to the function of the coating but may aid in improving the commercial utility.

The core-shell polymer is designed to be removable under alkaline conditions such as laundering. It has been formulated as a dilute spray solution that can be sprayed on towels or athletic wear. Towels have been evaluated by pet groomers and found not to develop “doggy odor” over the course of a week. This reduced the amount of laundry required and reduced the amount of water used for this purpose. The spray had been sprayed on horse blankets and found to reduce the odor from a sweaty horse. No irritation or other ill effects were seen on the horse or rider. After a couple weeks of riding the blanket had become dirty, but not odiferous, and then required washing. It likewise has been found that spraying athletic wear, if not stored in a dark place thereafter, with this allows clothing to be worn for workouts all week without building an objectionable odor. Untreated garments required washing after each use.

In one embodiment, the polymer is placed on fabrics such as washing and wiping cloths, diapers, sanitary napkin covers, hospital gowns, surgical drapes, sheets, pillow cases, curtains, backing material for garments, table cloths, bed spreads, sponges, underpads, etc. For these products and products such as sheets, pillow cases, hospital gowns and surgical drapes in particular, it is highly desirable to render the textiles or other type materials antimicrobial and/or antiviral. Indeed, the passage of liquid through surgical drapes, which are used during surgical procedures to isolate the patient from the operating room personnel and environment, is one source of bacterial contamination.

Certain embodiments have advantages in that the polymers are safer in both application and administration. For example, a polymer may be large enough to hinder absorption by cells. Further, a polymeric form may be safer to handle over a dust form. When the polymer containing the Rose Bengal is bonded to fabric or a substrate, there can be less migration of the Rose Bengal from the substrate and less leach out either in use or after disposal.

In another embodiment, the polymer or coating may be applied to animals, animal quarters, animal-traveled areas, and ill animals to treat and prevent microbial agents/infections; such as from bacteria, viruses and fungi.

There are several advantages of singlet oxygen anti-microbial technology cited. No heavy metals like silver, copper or mercury are involved. The treated substrates are inert and harmless to humans. A disadvantage of photo-catalytically generating singlet oxygen is that it requires light. It does not work in the dark. Other advantages of certain polymers is the polymers may be manufactured safer and more efficiently and use effectively a 10^{th} to 100th the amount of dye to produce optimal activity.

In certain embodiment, the second monomer can be an acrylic acid, methyl methacrylate (MMA), Methacrylic acid, Ethyl methacrylate (EMA), and/or n-Butyl methacrylate (BMA). Suitable Acrylates include Acrylic acid, 4-Acryloylmorpholine, [2-(Acryloyloxy)ethyl]trimethylammonium chloride, 2-(4-Benzoyl-3-hydroxyphenoxy)ethyl acrylate, Benzyl 2-propylacrylate, 2-Butoxyethyl acrylate, Butyl acrylate, tert-Butyl acrylate, 2-[(Butylamino)carbonyl]oxy]ethyl acrylate, tert-Butyl 2-bromoacrylate, 4-tert-Butylcyclohexyl acrylate, 2-Carboxyethyl acrylate, 2-Carboxyethyl acrylate oligomers anhydrous, 2-(Diethylamino)ethyl acrylate, Di(ethylene glycol)ethyl ether acrylate technical grade, Di(ethylene glycol) 2-ethylhexyl ether acrylate, 2-(Dimethylamino)ethyl acrylate, 3-(Dimethylamino)propyl acrylate, Dipentaerythritol penta-/hexa-acrylate, 2-Ethoxyethyl acrylate, Ethyl acrylate, 2-Ethylacryloyl chloride, Ethyl 2-(bromomethyl)acrylate, Ethyl cis-(-cyano)acrylate, Ethylene glycol dicyclopentenyl ether acrylate, Ethylene glycol methyl ether acrylate, Ethylene glycol phenyl ether acrylate, Ethyl 2-ethylacrylate, 2-Ethylhexyl acrylate, Ethyl 2-propylacrylate, Ethyl 2-(trimethyl silylmethyl)acrylate, Hexyl acrylate, 4-Hydroxybutyl acrylate, 2-Hydroxyethyl acrylate, 2-Hydroxy-3-phenoxypropyl acrylate, Hydroxypropyl acrylate, Isobornyl acrylate, Isobutyl acrylate, Isodecyl acrylate, Isooctyl acrylate, Lauryl acrylate, Methyl 2-acetamidoacrylate, Methyl acrylate, Methyl a.-bromoacrylate, Methyl 2-(bromomethyl)acrylate, Methyl 3-hydroxy-2-methylenebutyrate, Octadecyl acrylate, Pentabromobenzyl acrylate, pentabromophenyl acrylate, Poly(ethylene glycol)methyl ether acrylate, Poly(propylene glycol) acrylate, Poly(propylene glycol)methyl ether acrylate Soybean oil, epoxidised acrylate, 3-Sulfopropyl acrylate potassium salt, Tetrahydrofurfuryl acrylate, 3-(Trimethoxysilyl)propyl acrylate, 3,5,5-Trimethylhexyl acrylate. It was found that nitrogen-containing monomers such as acrylamide, acrylonitrile and N,N-dimethyl acrylamide could quench the singlet oxygen.

Preferably methyl methacrylate, methacrylic acid, and/or Butyl acrylate (BMA) are used. Acrylamides: 2-Acrylamidoglycolic acid, 2-Acrylamido-2-methyl-1-propanesulfonic acid, 2-Acrylamido-2-methyl-1-propanesulfonic acid sodium salt solution, (3-Acrylamidopropyl)trimethylammonium chloride solution, 3-Acryloylamino-1-propanol solution purum, N-(Butoxymethyl)acrylamide, N-tert-Butylacrylamide, Diacetone acrylamide, N,N-Dimethylacrylamide, N-[3-(Dimethylamino)propyl]methacrylamide, N-Hydroxyethyl acrylamide, N-(Hydroxymethyl)acrylamide, N-(Isobutoxymethyl)acrylamide, N-Isopropylacrylamide, N-Isopropylmethacrylamide, Methacrylamide, N-Phenylacrylamide, N-[Tris(hydroxymethyl)methyl]acrylamide.α

In certain embodiments, the first monomer can be a styrene operatively connected to Rose Bengal. These include Styrene, Divinyl benzene, 4-Acetoxystyrene, 4-Benzyloxy-3-methoxy styrene, 2-Bromostyrene, 3-Bromostyrene, 4-Bromostyrene, .a.-Bromostyrene, 4-tert-Butoxystyrene, 4-tert-Butyl styrene, 4-Chloro-.α.-methyl styrene, 2-Chlorostyrene, 3-Chloro styrene, 4-Chlorostyrene, 2,6-Dichlorostyrene, 2,6-Difluorostyrene, Dimethyl styrene, 1,3-Diisopropenylbenzene, 3,4-Dimethoxystyrene, α.,2-2,4-Dimethyl styrene, 2,5-Dimethyl styrene, N,N-Dimethylvinylbenzylamine, 2,4-Diphenyl-4-methyl-1-pentene, 4-Ethoxystyrene, 2-Fluorostyrene, 3-Fluorostyrene, 4-Fluorostyrene, 2-Isopropenylaniline, 3-Isopropenyl-α.,α.-dimethylbenzyl isocyanate, Methylstyrene, .α.-Methyl styrene, 3-Methylstyrene, 4-Methyl styrene, 3-Nitrostyrene, 2,3,4,5,6-Pentafluorostyrene, 2-(Trifluoromethyl)styrene, 3-(Trifluoromethyl)styrene, 4-(Trifluoromethyl)styrene, 2,4,6-Trimethylstyrene. Preferably Styrene is used.

In certain embodiments, the polymers can be vinyl Groups. For example, 3-Vinylaniline, 4-Vinylaniline, 4-Vinylpyridine, 4-Vinylanisole, 9-Vinylanthracene, 3-Vinylbenzoic acid, 4-Vinylbenzoic acid, Vinylbenzyl chloride, 4-Vinylbenzyl chloride, (Vinylbenzyl)trimethylammonium chloride, 4-Vinylbiphenyl, 2-Vinylnaphthalene, 2-Vinylpyridine, N-Vinyl-2-pyrrolidinone, 2-Vinylnaphthalene, Vinyl acetate, Vinyl benzoate, Vinyl 4-tert-butylbenzoate, Vinyl chloroformate, Vinyl chloroformate, Vinyl cinnamate, vinyl decanoate, vinyl neodecanoate, vinyl neononanoate, vinyl pivalate, vinyl propionate, vinyl stearate, and vinyl trifluoroacetate.

Other monomers which may be used are those which have groups to help stabilization of the particles, e.g. Poly(ethylene glycol)methyl ether acrylate, Poly(ethylene glycol)phenyl ether acrylate, lauryl methacrylate, Poly(ethylene glycol)methyl ether acrylate, Poly(propylene glycol)methyl ether acrylate, Lauryl acrylate and fluorinated monomers of above.

Other embodiment may include the use of anionic dyes and cationic dyes that are capable of generating singlet oxygen. Anionic dyes that provide a source of singlet oxygen include fluorescein derivatives (e.g.,), preferably the alkali metal salts of Rose Bengal, 4,5,6,7-tetrachloro-2′,5′,7′-tetraiodo fluorescein sodium or potassium (hereinafter, reference to Rose Bengal means an alkali metal salt thereof). Cationic dyes may include 3-amino-7-(dimethylamino)-2-methylphenothiazin-5-ium chloride (also known as Tolonium chloride or Toluidine Blue O), Thionin and Methylene Blue. Another dye may be tetraphenylporphyrin. The monomer is created by operatively linking the dyes by the respective carboxylic acid/salt group. The dyes may be used alone or in combination.

Other optional ingredients include, water, an amount of a suitable noncopolymerizable surfactant, thickeners, hiding pigments, opacifiers, colorants, antioxidants, biocides or any other ingredients typically added to latex polymers. Optional ingredients include conventional dispersants. These additional ingredients are not critical to the function of the coating but may aid in improving the commercial utility.

Working examples of preferred embodiments, including multiple photosensitizers, are provided below. As Shown below in another embodiment of the invention, photosensitizers have been applied to a fabric substrate, such as a mask for covering the mouth and nose of a user, by means of a water-based polymer. The photosensitizers are present in an amount sufficient to impart a characteristic color to the substrate, and in the presence of visible light, catalyze formation of singlet oxygen in therapeutically useful quantities for treating respiratory infections and cancers by inhalation of the singlet oxygen.

In one preferred non limiting embodiment, the fabric may be fashioned into a surgical type mask to facilitate inhalation of the singlet oxygen. The photosensitizer may be chemically modified to enable co-polymerization to become incorporated into a water-based polymer. If the water-based polymer is anionic, a cationic photosensitizer may also be applied to the fabric substrate by ionic attraction to the polymer.

A nonionic photosensitizer such as curcumin, contained in a natural matrix such as turmeric may be applied to the fabric substrate and enhanced by bonding of the matrix by a suitable water-based polymer. Thus, curcumin may be applied to fabric from a turmeric dispersion, bonded by an anionic water-based polymer containing a photosensitizer, and a cationic photosensitizer may be exhausted onto the substrate by ionic attraction to the polymer. Thus, 3 photosensitizers may be applied to the fabric. The fading of the characteristic color of the substrate is indicative that the photosensitizer has photodegraded to a level where generation of singlet oxygen has declined to a level where a new mask is recommended for further treatment.

Singlet Oxygen has a very short lifetime in aqueous environments such as blood, mucus or plasma, and doesn't travel far. But people suffering from Long Covid claim that wearing a pink mask constructed in accordance with the invention and inhaling the resulting air enriched with Singlet Oxygen gives them benefit. It is permissible to say the means and mechanism by which they benefit appears to be unclear at the moment, and much of Long Covid's effects and actions also remain unclear. But, without intending to be bound by theory, it is believed from empirical results that Singlet Oxygen may reduce or eliminate any low grade infection, inflammation or irritation resulting from residual lung tissue damage caused by an earlier Covid-19 infection. The oxidative action of Singlet Oxygen on damaged tissue may elicit a healing effect brought about by engaging the body's immune system. This explanation may bridge the gap between Long Covid benefits and what we know about Singlet Oxygen.

In accordance with the invention a cationic photosensitizer (such as Methylene Blue) is applied to a fabric treated with highly anionic polymer (solution or core shell). This would add to the photosensitizer loading and use a different wavelength of the incident light. It would be durable due to ionic bonding. It could be applied to the fabric by spray, dip or foam. Dipping would probably have differential absorption with initial fabric being heavily loaded and later fabric having a weak loading from a depleted bath. Spray or foam finishing would probably have more uniform application. As a result there are two photosensitizers at work in this embodiment.

In a preferred embodiment of the invention, a natural photosensitizer (curcumin) from turmeric, is applied as is, and with binder components such as starch, insolubilizer and sizing agent/water repellent to fabric substrates. It is well within the scope of the invention to use other natural photosensitizer sources to cover the base

### EXAMPLES

### Example 1

A roll of nonwoven polyester fabric, 46 cm wide, 38.2 g/m² was treated with a water-based, anionic, cross linkable solution polymer containing 1% (by dry weight) of a vinyl, benzyl derivative of Rose Bengal copolymerized into the polymer. The application was by a dip and nip process. The polymer was applied from an aqueous solution, dried and cured at 150° C. to achieve 0.5% dry add-on. The fabric was bright pink in color. The fabric was wound up on a roll and wrapped in black plastic for later use.

### Example 2

A surgical-type ear loop mask was obtained from commercial sources. The center portion was cut away leaving only the bonded frame around the edges. A bead of hot melt adhesive was applied around this bonded frame. A piece of pink fabric from the roll described in Example 1 measuring 17.6 cm by 9.5 cm was cut from the roll and fitted over the adhesive layer on the bonded frame. A hot iron was moved around the edge on the frame on the fabric to melt the glue and adhere the fabric to the bonded frame This is the general procedure for making a single layer prototype mask using fabric treated with one or more photosensitizers. The procedure could be modified to make a similar mask with one or more layers on a commercial pilot or production machine.

### Example 3

A piece of fabric 46 cm by 61 cm, 10.71 g, was cut from the pink fabric from Example 1. The fabric was tested for production of Reactive Oxygen Species (ROS), such as singlet oxygen, hydroxyl radical, hydrogen peroxide, by a UV/VIS method described by Gang Sun et al., Mater. Adv., 2021, 2, 3569-3578. The pink fabric was rated at 3.05 nanomoles/mg of fabric sample. A 0.1% solution of Methylene Blue was prepared in deionized water. The pink fabric was immersed in the Methylene Blue solution and thoroughly wetted. It was then squeezed out by hand as much as possible and air dried. The dried fabric was dark blue, 10.74 g. The fabric was washed in warm water to remove the excess blue dye. The dried fabric was now purple in color, 10.72 g. The resulting fabric was tested by the above method and found to be rated at 3.40 nanomoles/mg fabric sample for an increase of 11.5%. The fabric was cut into 4 pieces 9.5 cm by 17.5 cm, and made into masks as described in Example 2. The anionic polymer applied in Example 1 attracted the cationic Methylene Blue dye and held it in place by ionic attraction. This mask contains two photosensitizers.

### Example 4

A piece of untreated polyester fabric used in Example 1, measuring 46 cm by 61 cm, weighing 10.22 g was treated with a 3% mixture of an anionic core-shell emulsion polymer containing 0.08% vinyl benzyl Rose Bengal derivative in the shell. This core-shell polymer was opaque light pink in color. The fabric was squeezed out as much as possible and weighed 25.84 g for 153% wet pick up. The fabric was air dried and appeared a very pale shade of pink in color. Calculations show that 0.072 g dry latex is disposed on 2787 cm² of fabric. This fabric was tested for production of reactive Oxygen Species (ROS) by the same method used in Example 3 and found to produce 1.33 nanomoles/mg of fabric sample. This fabric was dipped in the 0.1% Methylene Blue solution and thoroughly wetted, then squeezed out and air dried. It was light blue in color. This fabric was tested for production of ROS and rated at 1.83 nanomoles/mg of fabric sample for an increase of 37.6%. This fabric contains 2 photosensitizers. Untreated fabric dipped in the Methylene Blue solution, squeezed and air dried was a very faint blue color.

### Example 5

A 1% dispersion of turmeric was prepared from 2.0 g of organic turmeric spice and 198 g of deionized water. This was stirred and heated to a boil, then quickly cooled. Upon reaching 40° C., the dispersion was passed through a hand-pumped homogenizer three times affording an opaque yellow dispersion. A fine orange precipitant formed upon standing. This was stirred and 1.2 g (20% active) of the solution polymer of Example 1 was added as a binder for the turmeric. The yellow mixture was poured into a tray and a piece on the untreated nonwoven fabric of Example 1 was passed through the mixture, then squeezed out for 166% wet pick up. The fabric was air dried. The fabric was bright yellow in color and contains 2 photosensitizers, curcumin from the turmeric and Rose Bengal from the polymer. The fabric was immersed in a dilute solution (0.01%) Methylene Blue and warmed to 50° C. for 10 minutes. The fabric absorbed Methylene Blue from the solution and was squeezed out and air dried. It was green in color, and contains three photosensitizers. The fabric was made into masks as described in Example 2.

### Example 6

A 1% dispersion of turmeric was prepared from 2.0 g of organic turmeric spice and 198 g of deionized water. This was stirred and heated to a boil, then quickly cooled. Upon reaching 40° C., the dispersion was passed through a hand-pumped homogenizer 3 times affording an opaque yellow dispersion. A fine orange precipitant formed upon standing. The yellow mixture was poured into a tray and a piece on the untreated nonwoven fabric of Example 1 was passed through the mixture, then squeezed out for 175% wet pick up. The fabric was air dried. The fabric was bright yellow in color and contained 1 photosensitizer, curcumin from the turmeric. The fabric was tested for Reactive Oxygen Species (ROS) by the method described in previous examples and was rated at 2.58 nanomoles ROS/mg fabric.

### Example 7

Sam L, a male, mid 40s, smoker since his teens, was diagnosed with COPD and emphysema, experiencing shortness of breath. He developed recurring lung infections with much congestion, which made breathing difficult. The antibiotic Cipro® was prescribed. The infection cleared, but would soon return. The cycle repeated until Cipro® was no longer effective. His condition degenerated until he had to quit work and was mostly bed bound. He was offered a mask made with the pink fabric described in Example 1 and instructed in its use. He used a tanning bed as a light source and did 5 or 6 sessions a day wearing the mask, each session lasting 10 to 15 minutes. He felt no benefit for the first 3 days. By day 5 he was feeling better and breathing easier. By day 10 he was feeling well and getting out to run errands. A visit with his doctor on day 14 showed by x-ray his lungs were clear and his respirometer reading was the best since his diagnosis. He has remained free of this recurring infection for over 5 years at the time of this writing.

### Example 8

Tom H, a male, mid 60s and diabetic developed an ulcer on his foot. It became infected with MRSA. The infection spread to his lungs. He was treated with various antibiotics administered by a PICC line over 3 months, with no success. He was given a mask made from the treated fabric described in Example 1, and instructed in its use. He saw no benefit for the first 5 days. On day 6 he noted some improvement. By day 10, he felt normal with no congestion. He has remained clear of this infection for over one year.

### Example 9

David K was a terminal cancer patient who brought in hospice for end-of-life care. He had a MRSA type lung infection and had been treated with Levaquin®, but it was discontinued as it was showing no benefit. He was on oxygen around the clock and moved around the house with great difficulty. He was made aware of the mask technology and requested a mask. His wife obtained a 100 watt incandescent light bulb and placed it in a lamp that was positioned by his face when he was wearing the pink mask. This bulb caused too much heat on his face, so she replaced it with a comparable LED bulb. In 3 days he started feeling better. By day 5 he reduced his oxygen flow rate. By day 10 he was able to sit outside and enjoy the flowers and birds in his yard. He was now able to take his wife out for dinner one last time. He estimated he experienced a 35% improvement in his breathing ability. He soon passed from his cancer, but he and his wife were grateful for the brief improvement in his quality of life at the end.

### Example 10

Kristie K was a female, 49, smoker diagnosed as terminal, with stage 4 small cell lung carcinoma in April, 2020. She had tumors in her lungs, bronchial tube, liver, kidney, adrenal gland and upper right arm bone. She was told she′d be lucky to make it to the end of the year, and to get her affairs in order. The only treatment offered as a last resort was Keytruda®. She had an adverse reaction after 1 dose and almost died. The tumor in her arm caused the bone to fail and a rod was inserted to stabilize it. She had 15 radiation treatments on her arm. Her tumors continued to advance.

In December 2020, she was offered a mask as described in Example 2 and instructed on its use. It was thought it may prevent an opportunistic respiratory infection from hastening her demise. She wore the mask as much as possible during the day. After a week, she started feeling better, more energy, strength and appetite. By end of the month she had gained 5 pounds. In January, a CT scan showed no change from the scan the previous October. In February, the pink color of the mask had noticeably faded, and a new one provided. A CT scan in April showed the tumors were shrinking, and were smaller than in January. As the mask faded again, a new one was provided. A CT scan in July showed further shrinkage. As the color of the mask had faded, a new one was provided. A CT scan in October showed little further shrinkage, but her lab test results were now within normal ranges. As of July 2022, her tumors remain shrunken and appear to be in a dormant state. Her lab test results remain in the normal range. Her oncologist is surprised at her recovery and advised her to enjoy life, live it to the fullest and come back in 3 months. as of the filing date hereof she continues to use the pink mask and replace it when the color fades.

### Example 11

In November, 2020, William F., male, 66, non-smoker and considered “high risk” for experiencing severe, if not fatal, symptoms due to age, heart condition (A-fib) and recent cancer treatment, awoke with a slight fever, body ache, and light cough. He recognized this as symptoms of Covid-19 and began wearing a mask as described in Example 2, and isolated himself. The body ache ceased after a couple of hours. He wore the same mask during waking hours for the next 10 days. That evening his temperature reached a high point of 99.9° F. Morning temperatures were subnormal, and the high point for the day declined over the next 4 days until achieving a normal reading of 98.4° F. The cough also vanished. He felt well and normal. He came out of isolation after a week. Two months later, a blood test revealed no active Covid-19 virus, but Covid-19 antibodies were present, indicating he had Covid-19 earlier, successfully treated it with the mask described in Example 2 experiencing very mild symptoms, and recovered. No other treatment or medications were employed

### Example 12

Janice D., a female, in her 80s, non-smoker, was diagnosed with COPD, experiencing shortness of breath, She developed a severe respiratory infection with much congestion, which made breathing difficult. She was coughing up sputum green or dark brown in color. Her doctor put her on doxycycline for two weeks. She felt better while on the antibiotics, and the sputum became less in amount and lighter in color. After she finished the antibiotics, within 3 days, her breathing became labored again and sputum darkened and increased in amount. She was offered a mask made with the pink fabric described in Example 2 and instructed in its use. She used the pink mask several times per day at home turning her face towards the sun. Within a few days of using mask, the sputum that usually looked dark, lightened significantly. She was able to breathe much better and her energy came back She became more active. As the pink began to fade, her symptoms became worse again. Upon replacement with another mask, within 2 to 3 days her symptoms were noticeably better. Her energy came back. She had been using the masks for 4 years. The last mask seemed to have lost its effect after she′d been using it for almost a year. Her decline became noticeable within less than a week when she was not using the mask. She passed away Jul. 29, 2021 and she never had another respiratory infection since she began using the mask. She affirmed that the mask improved her quality of life.

### Example 13

The pink fabric made in Example 1 was originally tested by the method described above by Gang Sun et al as having Reactive Oxygen Species (ROS) activity as 3.05 nanomoles ROS/mg fabric. The fabric was made into masks for long term use as described in Example 2. After approximately two months of daily use, the bright pink color of the mask had faded to a very pale pink color. The faded fabric was tested for ROS activity. It tested at 1.14 nanomoles ROS/mg fabric, thus demonstrating a significant (approximately 63%) decline in activity associated with fading of the original characteristic color. The degree of fade is determined by the observer and may vary from sample to sample.

It is believed that singlet oxygen as used in accordance with the invention, oxidizes functionalities of the surface of bacteria, thus rendering them non-viable. Thus, they have no opportunity to develop resistance mechanisms, as they have done for antibiotics, which must be ingested to be effective. By a similar process, various fungi have developed resistance to commonly used fungicides. Currently, only a small number of antifungal drug types exist, so resistance can severely limit treatment options. Some types of fungi, like Candida auris, can become resistant to all the antifungal drugs normally used to treat these infections. Resistance is especially concerning for patients with invasive fungal infections—severe infections that affect the blood, heart, brain, eyes, or other parts of the body.

Some species of fungi are naturally resistant to certain types of antifungal drugs. For example, the drug fluconazole does not work against infections caused by the fungus Aspergillus, a type of mold found throughout the environment. Resistance can also develop over time when fungi are exposed to antifungal drugs. This resistance can occur when antifungal drugs are used to treat sick people, especially if the drugs are used improperly (for example, when dosages are too low or when treatment courses are not long enough).

Use of fungicides in agriculture to prevent and treat fungal diseases in crops can also contribute to resistant disease in people. For example, when Aspergillus found in the environment is exposed to fungicides, which are similar to medical antifungal drugs, the Aspergillus can become resistant to the drugs used to treat infections in people. People can then breathe in those resistant Aspergillus spores from the environment and become sick.

Resistant fungi include Aspergillus, certain Candida species, and certain dermatophytes. Candida auris is a newer species of Candida that is particularly resistant to antifungal drugs and can spread quickly in healthcare settings. It is thought that singlet oxygen can likewise oxidize external functional groups on the surface of fungus cells and render them non-viable while not providing an opportunity to develop a resistance mechanism. It would seem that singlet oxygen as provided by this invention would be useful against topical/external and respiratory fungal infections.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others. Unless specifically recited in a claim, steps or components of claims should not be implied or imported from the specification or any other claims as to any particular order, number, position, size, shape, angle, color, or material.

What is claimed:

1. A surgical type face mask comprising:
a substrate, the substrate having an outer layer, the outer layer being treated with a water-based polymer containing at least one photosensitizer in sufficient quantity to impart a characteristic color, and in the presence of visible light, the water based polymer generating therapeutically useful quantities of singlet oxygen for treatment, by inhalation thereof, of respiratory infections and cancers.

2. The face mask of claim 1, in which the characteristic color fades due to photodegradation, the color fading being indicative of the amount of singlet oxygen generated being insufficient for effective treatment of respiratory infection or cancer.

3. The face mask of claim 1 in which the photosensitizer is derived from Rose Bengal.

4. The face mask of claim 1 in which the photosensitizer is Methylene Blue.

5. The face mask of claim 1 in which the outer layer has been treated with a dispersion of turmeric, the turmeric containing a curcumin, the curcumin being the photosensitizer, and the water-based polymer is anionic and contains Rose Bengal.

6. The face mask of claim 1 in which the water-based polymer is anionic, contains Rose Bengal, and a cationic Methylene Blue has been exhausted onto the substrate.

7. The face mask of claim 5 in which the substrate treated with the turmeric dispersion and the water-based polymer is anionic, contains Rose Bengal and Methylene Blue, the Methylene Blue being exhausted onto the substrate, resulting in nonionic, anionic and cationic photosensitizers being attached to the substrate, the nonionic, anionic and cationic photosensitizers each a different color and having different absorbance maxima in the visible light spectrum.
